(19) United States
(12) Patent Application Publication
(10) Pub. No.: US 2012/0165654 A1
(43) Pub. Date: Jun. 28, 2012

(54) MEANS AND METHOD FOR PERFORMING HYPERPOLARIZING GAS IMAGING

(75) Inventor: Uri Rapoport, Moshav Ben Shemen (IL)

(73) Assignee: ASPECT MAGNET TECHNOLOGIES LTD., Shoham (IL)

(21) Appl. No.: 13/392,162

(22) PCT Filed: Aug. 25, 2010
(86) PCT No.: PCT/IL10/00691

§ 371 Date: Mar. 15, 2012

Related U.S. Application Data
(60) Provisional application No. 61/236,879, filed on Aug. 26, 2009.

(51) Int. Cl. A61B 5/055; A61B 6/00; G01N 27/72; G01N 21/17; G01N 21/64; A61B 5/00; C12M 1/34

(52) U.S. Cl. 600/411; 600/431; 600/420; 600/425; 435/288.7; 435/29

(57) ABSTRACT

The present invention discloses a system for hyperpolarizing un-polarized gas within an animal. The system comprises hyper polarization means for hyperpolarizing the un-polarized gas, wherein the hyperpolarization of the un-polarized gas is provided in-situ within the analyzed animal.

### FIELD OF THE INVENTION

This invention generally relates to a device used for performing hyperpolarizing gas imaging. Furthermore, the device of the present invention provides means for providing the hyperpolarizing gas in situ.

### BACKGROUND OF THE INVENTION

Nuclear magnetic resonance imaging (MRI) is an important modality for both clinical and basic-science imaging applications. A recent notable advance in MRI was the introduction of the “hyperpolarized” noble gases helium-3 (³He) and xenon-129 (¹²⁹Xe) as novel magnetic-resonance contrast agents.

Nuclear polarization levels approaching 100 percent can be achieved using hyperpolarized noble gases, and this dramatic increase in the polarization compared to that typically achieved at thermal equilibrium (at most approximately 10-4) has presented the opportunity for many new MRI applications.

For example, high-resolution MR images of the lung air spaces have been demonstrated following the inhalation of hyperpolarized-³He gas, and studies suggest that ³He lung imaging shows promise for differentiating healthy lungs from those with pathologies such as chronic obstructive pulmonary disease, asthma and cystic fibrosis. Therefore, it would be beneficial to provide a device and method that perform hyperpolarizing gas imaging while producing the hyperpolarized gas in situ.

### SUMMARY OF THE INVENTION

It is thus one object of the present invention to provide a system for hyperpolarizing un-polarized gas within an animal, comprising hyper polarization means for hyperpolarizing the un-polarized gas, wherein the hyperpolarization of the un-polarized gas is provided in-situ within the animal.

It is another object of the present invention to provide the system as defined above, wherein the hyper polarization means is selected from laser, ultrasound, RF, microwave, application of heat or any combination thereof.

It is another object of the present invention to provide the system as defined above, wherein the gas is selected from helium or Xenon.

It is another object of the present invention to provide the system as defined above, wherein the animal is selected from a group consisting of mammal, humans, premature babies, reptiles, sea animals, biological specimens, biological organs, mice, rats, rodents, birds, reptiles, amphibians, in vivo biological tissue or organ or ex vivo biological tissue or organ.

It is another object of the present invention to provide a system for hyperpolarizing un-polarized gas confined within a volume, the volume having a medium therein, comprising at least one volume confining an un-polarized gas and at least one medium; and hyper polarization means for hyperpolarizing the un-polarized gas; wherein the hyperpolarization of the un-polarized gas is provided in vitro within the confined volume.

It is another object of the present invention to provide the system as defined above, wherein the hyper polarization means is selected from laser, ultrasound, RF, microwave, application of heat or any combination thereof.

It is another object of the present invention to provide the system as defined above, wherein the gas is selected from helium or Xenon.

It is another object of the present invention to provide the system as defined above, additionally comprising a chamber in fluid communication with the volume, the chamber accommodates at least one animal, such that the hyperpolarized gas is supplied from the volume to the chamber.

It is another object of the present invention to provide the system as defined above, wherein the animal is selected from a group consisting of mammal, humans, premature babies, reptiles, sea animals, biological specimens, biological organs, mice, rats, rodents, birds, reptiles, amphibians, in vivo biological tissue or organ or ex vivo biological tissue or organ.

It is another object of the present invention to provide the system as defined above, wherein the medium is selected from a group consisting of anesthetic gas, water, oxygen or any combination thereof.

It is another object of the present invention to provide the system as defined above, wherein anesthetic gas, water, oxygen or any combination thereof is supplied to the chamber

It is another object of the present invention to provide a system for hyperpolarized gas imaging of at least one animal, comprising: at least one volume confining an un-polarized gas and at least one medium; at least one chamber confining a volume in size and shape for accommodating the at least one animal; the chamber is in fluid communication with the volume; supplying mechanism for supplying un-polarized gas to the at least one volume; hyper polarization means for hyperpolarizing the un-polarized gas; and, imaging device for imaging at least a region of the animal;

wherein the hyperpolarization of the un-polarized gas is provided in vitro within the confined volume.

It is another object of the present invention to provide a system for hyperpolarized gas imaging of at least one animal, comprising: at least one chamber confining a volume in size and shape for accommodating the at least one animal; supplying mechanism for supplying un-polarized gas to the at least one chamber; hyper polarization means for hyperpolarizing the un-polarized gas; and, imaging device for imaging at least a region of the animal; wherein the hyperpolarization of the un-polarized gas is provided within the confined volume.

It is another object of the present invention to provide the systems as defined above, wherein the imaging device is selected from a group consisting of NMR, MRI, CT, X-ray, ultrasound device, fluorescence device, thermographic device or any combination thereof.

It is another object of the present invention to provide the systems as defined above, wherein the medium is selected from a group consisting of anesthetic gas, water, oxygen or any combination thereof.

It is another object of the present invention to provide the systems as defined above, wherein the hyper polarization means is selected from laser, ultrasound, microwave, RF, application of heat or any combination thereof.

It is another object of the present invention to provide the systems as defined above, wherein the gas is selected from helium or Xenon.

It is another object of the present invention to provide the systems as defined above, wherein the animal is selected from a group consisting of mammal, humans, premature babies, reptiles, sea animals, biological specimens, biological organs, mice, rats, rodents, birds, reptiles, amphibians, in vivo biological tissue or organ or ex vivo biological tissue or organ.

It is another object of the present invention to provide the systems as defined above, wherein the imaging device is selected from a group consisting of NMR, MRI.

It is another object of the present invention to provide the systems as defined above, wherein the NMR/MRI system comprising a spatially fixed coupled imaging device (SFCID) for producing combined anatomical and real time functional light images, the SFCID functionally incorporates a maneuverable imaging system MIS with a coupled imaging system CIS: the maneuverable imaging system (MIS) contains an imaging platform (IMP) accommodating an immobilized subject positioned within a nonconductive housing; the IMP is contained within a radio frequency coil system (RFCS) for imaging one or more regions of a subject; the RFCS is adapted either to reversibly translate (i) at least one conductive receiver coil, and/or (ii) at least a portion of the IMP, in at least one nonconductive housing coil to at least one fixed position to an accuracy of not less than about 3 mm while the subject remains within the MIS; the RFCS includes: a mechanical translation system (MTS) adapted for providing linear motion to the immobilized subject and for reproducibly fixing the position of the immobilized subject to within a range of about 3 to about 60 mm; and, attaching means (AM) for connecting the housing to the MTS; and, the coupled imaging system (CIS) adapted to image at least one specific region of the immobilized subject, and to integrate (i) at least one MRD imaging module (MIM) configured for providing three dimensional anatomical images; with (ii) at least one optical imaging module (OIM), coupled to the IMP and configured for detecting photons emitted or reflected by the region of the immobilized subject so as to generate real time functional light images of a functionally active part of the region of the immobilized subject; the functional incorporation of coupled MIM and OIM in the IMP provides one or more multi-modular fused, real-time images of the region of the immobilized subject located within a determinable specific volume.

It is another object of the present invention to provide the systems as defined above, wherein the RF coil is selected from the group consisting of a solenoid, a Helmholtz coil, and a surface coil.

It is another object of the present invention to provide the systems as defined above, further comprising a nonconductive housing which defines a volume of interest (VOI); a magnet adapted for generating a stable magnetic field with a defined magnetic field axis in the VOI; a plurality of coils adapted for establishing at least one magnetic gradient within the VOI; at least one non-conductive housing coil (NCHC) adapted for applying pulses of RF radiation to excite nuclear spins within the immobilized subject in the VOI; and, at least one conductive receiver coil (CRC) located within the NCHC; wherein the CRC is adapted to optimize reception of resonance signals emanating from the immobilized subject within a determinable specific volume provided within the VOI.

It is another object of the present invention to provide the systems as defined above, wherein at least one of the fixed positions is located outside of the nonconductive housing.

It is another object of the present invention to provide the systems as defined above, wherein one of the fixed positions is the point at which the optimized reception occurs at the point along the midpoint of the stable magnetic field along the magnetic field axis.

It is another object of the present invention to provide the systems as defined above, wherein, at least one of the fixed positions is located outside of the volume and one of the fixed positions is the point at which the optimized reception occurs at the point along the midpoint of the stable magnetic field along the magnetic field axis.

It is another object of the present invention to provide the systems as defined above, wherein the imaging platform (IMP) is a bad.

It is another object of the present invention to provide the systems as defined above, further comprising: a second mechanical translation system (MTS) adapted for providing linear motion to the immobilized subject and for reproducibly fixing the position of the immobilized subject within a range of about 3 mm to about 60 mm; and, attaching means (AM) for connecting the IMP or portions thereof to the MTS; wherein the IMP is adapted reversibly to translate relative to the determinable specific volume independent of the translation of the CRC.

It is another object of the present invention to provide the systems as defined above, wherein the AM adapted to connect the mechanical translation system (MTS) attached to the IMP with the MTS attached to the CRC, and further wherein the motions of the IMP and CRC are interdependent.

It is another object of the present invention to provide the systems as defined above, wherein the optical imaging module (OIM) comprises a plurality of detectors functionally incorporated within the perimeter of the housing; and, means for transmitting a signal from each of the plurality of detectors to a controller located external to the volume; wherein the functional incorporation of the plurality of detectors within the hosing enables production combined anatomical and real time functional light images.

It is another object of the present invention to provide the systems as defined above, wherein the optical imaging module (OIM) comprises a plurality of optic fibers functionally incorporated within the perimeter of the housing; and, means for transmitting a signal from each of the plurality of optic fibers to a controller located external to the volume; wherein the functional incorporation of the plurality of optic fibers within the hosing enables production combined anatomical and real time functional light images.

It is another object of the present invention to provide the systems as defined above, wherein the coupled imaging system (CIS) provides an imaging method selected from the group consisting of (a) fluorescence spectroscopy, (b) SPECT, (c) PET, and any combination of the above; and further wherein the plurality of either detectors and/or optics fibers are adapted for detecting signals typical of the at least one additional imaging method.

It is another object of the present invention to provide the systems as defined above, wherein spatially fixed coupled imaging device (SFCID) is adapted for 3-dimensional (3D) multimodal imaging.

It is another object of the present invention to provide the systems as defined above, wherein the device is provided with a self-fastening cage of a magnetic resonance device (MRD) (100) for providing a homogeneous, stable and uniform magnetic field therein, characterized by an outside shell comprising at least three flexi-jointed superimposed walls (1) disposed in a predetermined arrangement clockwise or counterclockwise.

It is another object of the present invention to provide the systems as defined above, wherein the MRD comprises: at least six side-magnets arranged in two equal groups being in a face-to-face orientation in a magnetic connection with the cage walls characterized by an outside shell comprising at least three flexi-jointed superimposed walls disposed in the same predetermined arrangement of the cage walls, increasing the overall strength of the magnetic field provided in the cage; at least two pole-magnet pieces, arranged in a face-to-face orientation in between the side-magnets; and, at least two main-magnets, located on the pole-pieces, arranged in a face-to-face orientation, generating the static magnetic field therein the cage.

It is another object of the present invention to provide the systems as defined above, comprising at least one Central Processing Unit (CPU) for processing and integrating the three dimensional MRD images received from the at least one MRD imaging module (MIM) and the real time functional light images received from the at least one optical imaging module (OIM).

It is another object of the present invention to provide the systems as defined above, wherein the CPU is provided with means to display the three dimensional MRD images and the real time light images.

It is another object of the present invention to provide the systems as defined above, wherein the CPU is provided with means for distinguishing the real time light images from the three dimensional NMR images of the region of the immobilized subject such that functionally active parts of the region of the immobilized subject are identifiable in real time.

It is another object of the present invention to provide the systems as defined above, wherein the MRD module comprises CT means.

It is another object of the present invention to provide the systems as defined above, wherein the MRD module comprises MRI means.

It is another object of the present invention to provide the systems as defined above, wherein the MRD module is provided with Two-Dimensional Fourier Transform (2DFT) means and slice selection means for building the image.

It is another object of the present invention to provide the systems as defined above, wherein the MRD module is provided with Three-Dimensional Fourier Transform (3DFT) means for building the image.

It is another object of the present invention to provide the systems as defined above, wherein the MRD module is provided with projection reconstruction means for building the image.

It is another object of the present invention to provide the systems as defined above, wherein the MRD module is provided with point by point image building means for building the image.

It is another object of the present invention to provide the systems as defined above, wherein the MRD module is provided with line by line image building means for building the image.

It is another object of the present invention to provide the systems as defined above, wherein the MRD module is provided with static field gradient image building means for building the image.

It is another object of the present invention to provide the systems as defined above, wherein the MRD module is provided with RF field gradient image building means for building the image.

It is another object of the present invention to provide the systems as defined above, wherein the optical imaging module comprises a light detector array including a plurality of light detectors distributed around the imaging platform in a predetermined manner for providing three dimensional real time light images of the region the immobilized subject.

It is another object of the present invention to provide the systems as defined above, wherein the optical imaging module is provided with means for detecting bioluminescence of the region of the immobilized subject.

It is another object of the present invention to provide the systems as defined above, wherein the optical imaging module is provided with means for detecting chemiluminescence of the region of the immobilized subject.

It is another object of the present invention to provide the systems as defined above, wherein the optical imaging module is provided with means for detecting fluorescence of the region of the immobilized subject.

It is another object of the present invention to provide the systems as defined above, wherein the optical imaging module is provided with means for detecting near infra-red fluorescence of the region of the immobilized subject.

It is another object of the present invention to provide the systems as defined above, wherein the optical imaging module includes means for single photon emission computed tomographic imaging (SPECT) of the region the immobilized subject.

It is another object of the present invention to provide the systems as defined above, wherein the optical imaging module includes means for Positron emission tomographic imaging. (PET) of the region of the immobilized subject,

It is another object of the present invention to provide the systems as defined above, wherein the optical imaging module includes photon counting sensitivity means.

It is another object of the present invention to provide the systems as defined above, wherein the optical imaging module includes means for selectively detecting excitation pulses traveling back from the region of the immobilized subject.

It is another object of the present invention to provide the systems as defined above, wherein the optical imaging module further includes means for synchronizing the excitation pulses.

It is another object of the present invention to provide the systems as defined above, wherein the immobilized subject is a small mammal.

It is another object of the present invention to provide the systems as defined above, wherein the immobilized subject is selected from a group consisting of humans, premature babies, mammals, biological specimens, biological organs, mice, rats, rodents, birds, reptiles, amphibians, in vivo biological tissue or organ or ex vivo biological tissue or organ.

It is another object of the present invention to provide a method for hyperpolarized gas imaging of at least one animal. The method comprises steps selected inter alia from: providing at least one chamber confining a volume in size and shape; accommodating the at least one animal within the at least one chamber; supplying un-polarized gas to the at least one chamber; hyperpolarizing the un-polarized gas; and, imaging at least a region of the animal whilst at least one region of the animal contains the hyperpolarized gas for at least a portion of the time required for the imaging; wherein the step of hyperpolarizing the un-polarized gas is performed within the confined volume.

It is another object of the present invention to provide the method as defined above, additionally comprising step of selecting the imaging device from a group consisting of NMR, MRI, CT, X-ray, ultrasound device, fluorescence device, thermographic device or any combination thereof.

It is another object of the present invention to provide the method as defined above, additionally comprising step of selecting the hyperpolarization means from laser, RF, ultrasound, microwave, application of heat or any combination thereof.

It is another object of the present invention to provide the method as defined above, additionally comprising step of selecting the animal from a group consisting of mammal, humans, premature babies, reptiles, sea animals, biological specimens, biological organs, mice, rats, rodents, birds, reptiles, amphibians, in vivo biological tissue or organ or ex vivo biological tissue or organ.

It is another object of the present invention to provide the method as defined above, additionally comprising step of selecting the gas from helium or Xenon.

It is another object of the present invention to provide the method as defined above, additionally comprising step of pausing the hyperpolarizing of the un-polarized gas during the step of imaging.

It is another object of the present invention to provide the method as defined above, additionally comprising step of producing combined anatomical and real time functional light images, by functionally incorporating a maneuverable imaging system MIS with a coupled imaging system CIS.

It is another object of the present invention to provide the method as defined, above, wherein the step of producing additionally comprising steps of: providing a spatially fixed coupled imaging device (SFCID) in a magnetic resonance imaging system, providing the MIS with an imaging platform (IMP), accommodating an immobilized subject positioned within a nonconductive housing; providing the IMP within a radio frequency coil system (RFCS) for imaging one or more regions of a subject; providing the RFCS with means to either reversibly translate (i) at least one conductive receiver coil (CRC), and/or (ii) at least a portion of the IMP, in at least one nonconductive housing coil (NCHC) to at least one fixed position to an accuracy of not less than about 3 mm while the subject remains within the MIS; further providing the RFCS with a mechanical translation system (MTS), and attaching means (AM) for connecting the housing to the MTS by means of the MTS, maneuvering the immobilized subject in a linear motion, and reproducibly fixing the position of the immobilized subject to within a range of about 3 to about 60 mm; imaging at least one specific region of the immobilized subject, by integrating (i) at least one MRD imaging module (MIM) configured for providing three dimensional anatomical images; with (ii) at least one optical imaging module (OIM), coupled to the IMP and configured for detecting photons emitted or reflected by the region of the immobilized subject thus generating real time functional light images of a functionally active part of the region of the immobilized subject; and, functionally incorporating MIM and OIM in the IMP, thus providing one or more multi-modular fused, real-time images of the region of the immobilized subject located within a determinable specific volume.

It is another object of the present invention to provide the method as defined above, comprising the steps of: introducing the immobilized subject to a determinable specific position within a stable magnetic field generated by a magnet; placing a positionable NCHC in proximity to the immobilized subject such that the position of the NCHC is fixed to within about 3 mm to about 60 mm and such that at least part of the volume of interest is located within the volume defined by the NCHC; exciting nuclear magnetization in the volume of interest by applying RF pulses and magnetic field gradients according to a predetermined imaging protocol; receiving RF imaging signals generated in the NCHC by the excited nuclear magnetization; and, reconstructing a magnetic resonance image of the determinable specific volume from the received magnetic resonance imaging signals and from the position of the NCHC.

It is another object of the present invention to provide the method as defined above, useful for optimizing reception of resonance signals emanating from the determinable specific volume, wherein the step of placing an NCHC in proximity to the object further includes a step of placing the NCHC at the point along the midpoint of the stable magnetic field along the magnetic field axis.

It is another object of the present invention to provide the method as defined above, comprising introducing the immobilized subject to a determinable specific position, the position located within a volume at least part of the interior of which contains stable magnetic field generated by a magnet and about the perimeter of which a plurality of detectors are disposed; placing a positionable RF receiver coil in proximity to the object such that the position of the RF receiver coil is fixed to within X mm and such that at least part of the volume of interest is located within the volume defined by the coil; exciting nuclear magnetization in the volume of interest by applying RF pulses and magnetic field gradients according to a predetermined imaging protocol; receiving RF imaging signals generated in the RF receiver coil by the excited nuclear magnetization; reconstructing a magnetic resonance image of the volume of interest from the received magnetic resonance imaging signals and from the position of the RF receiver coil; and, transmitting a signal from each of at least one of the plurality of detectors to a controller located external to the volume, the transmission commencing at a predetermined time relative to the commencement of step (c) and continuing for a predetermined length of time.

It is another object of the present invention to provide the method as defined above, wherein the at least one other imaging technique is selected from the group consisting of (a) fluorescence spectroscopy; (b) SPECT; (c) PET; and (d) any combination thereof.

It is another object of the present invention to provide a method for hyperpolarizing un-polarized gas within an animal. The method comprising steps of providing the animal at least partially containing the un-polarized gas; obtaining hyper polarization means for hyperpolarizing the un-polarized gas; hyperpolarizing the un-polarized gas; wherein the step of hyperpolarizing the un-polarized gas is performed in situ within the animal.

It is another object of the present invention to provide the method as defined above, additionally comprising step of selecting the hyper polarization means is selected from laser, ultrasound, RF, microwave, application of heat or any combination thereof.

It is another object of the present invention to provide the method as defined above, additionally comprising step of selecting the gas is selected from helium or Xenon.

It is another object of the present invention to provide the method as defined above, additionally comprising step of selecting the animal is selected from a group consisting of mammal, humans, premature babies, reptiles, sea animals, biological specimens, biological organs, mice, rats, rodents, birds, reptiles, amphibians, in vivo biological tissue or organ or ex vivo biological tissue or organ.

It is another object of the present invention to provide a method for hyperpolarizing polarized gas confined within a volume, the volume having a medium therein, the method comprising steps of: providing at least one volume confining an un-polarized gas and at least one medium; obtaining hyper polarization means for hyperpolarizing the un-polarized gas; hyperpolarizing the un-polarized gas; wherein the step of hyperpolarizing the un-polarized gas is performed in situ within the animal.

It is another object of the present invention to provide the method as defined above, additionally comprising step of selecting the hyper polarization means is selected from laser, ultrasound, RF, microwave, application of heat or any combination thereof.

It is still an object of the present invention to provide the method as defined above, additionally comprising step of selecting the gas is selected from helium or Xenon.

It is still an object of the present invention to provide the method as defined above, additionally comprising step of providing a chamber in fluid communication with the volume, the chamber accommodating at least one animal, such that the hyperpolarized gas is supplied from the volume to the chamber.

It is still an object of the present invention to provide the method as defined above, additionally comprising step of selecting the animal from a group consisting of mammal, humans, premature babies, reptiles, sea animals, biological specimens, biological organs, mice, rats, rodents, birds, reptiles, amphibians, in vivo biological tissue or organ or ex vivo biological tissue or organ.

It is another object of the present invention to provide method for hyperpolarized gas imaging of at least one animal. The method comprising steps selected from: providing at least one chamber confining a volume in size and shape; accommodating the at least one animal within the at least one chamber; providing at least one volume confining an un-polarized gas and at least one medium; supplying un-polarized gas to the at least one chamber; hyperpolarizing the un-polarized gas; and, imaging at least a region of the animal whilst at least one region of the animal contains the hyperpolarized gas for at least a portion of the time required for the imaging; wherein the step of hyperpolarizing the un-polarized gas is performed in vitro within the confined volume.

It is another object of the present invention to provide the method as defined above, additionally comprising step of selecting the imaging device from a group consisting of NMR, MRI, CT, X-ray, ultrasound device, fluorescence device, thermographic device or any combination thereof.

It is another object of the present invention to provide the method as defined above, additionally comprising step of selecting the hyperpolarization means from laser, RF, ultrasound, microwave, application of heat or any combination thereof.

It is another object of the present invention to provide the method as defined above, additionally comprising step of selecting the animal from a group consisting of mammal, humans, premature babies, reptiles, sea animals, biological specimens, biological organs, mice, rats, rodents, birds, reptiles, amphibians, in vivo biological tissue or organ or ex vivo biological tissue or organ.

It is another object of the present invention to provide the method as defined above, additionally comprising step of selecting the gas from helium or Xenon.

It is another object of the present invention to provide the method as defined above, additionally comprising step of pausing the hyperpolarizing of the un-polarized gas during the step of imaging.

It is another object of the present invention to provide the method as defined above, additionally comprising step of selecting the medium from a group consisting of anesthetic gas, water, oxygen or any combination thereof.

It is lastly an object of the present invention to provide the method as defined above, additionally comprising step of supplying the chamber with anesthetic gas, water, oxygen or any combination thereof.

### BRIEF DESCRIPTION OF FIGURES

In order to understand the invention and to see how it may be implemented in practice, a few preferred embodiments will now be described, by way of non-limiting example only, with reference to the accompanying drawings, in which:

FIG. 1a illustrates one embodiment of the present invention.

FIG. 1b presents a schematic diagram of a novel spatially fixed coupled imaging device (SFCID) useful for producing combined anatomical and real time functional light images. The SFCID functionally incorporates a maneuverable imaging system MIS with a coupled imaging system CIS according to an embodiment of the invention herein disclosed.

FIG. 2 presents a schematic diagram of an MRI system incorporating a positionable MRI receiver coil assembly according to an embodiment of the invention herein disclosed.

FIG. 3 presents a schematic diagram of an MRI system incorporating a positionable MRI receiver coil and independently movable bed according to an embodiment of the invention herein disclosed.

FIGS. 4a and 4b present a schematic diagram (side view and front view, respectively) of an MRI system incorporating a positionable MRI receiver coil and means for a second imaging method according to an embodiment of the invention herein disclosed.

FIG. 5 presents a schematic diagram of an integrated functional imaging modality and anatomical imaging modality according to an embodiment of the invention herein disclosed.

FIG. 6 presents a schematic diagram a method for acquiring integrated (fused) real-time (functional) image of immobilized non-moving subject according an embodiment of the invention herein disclosed.

### DETAILED DESCRIPTION OF THE PREFERED EMBODIMENTS

The following description is provided, alongside all chapters of the present invention, so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide means and method of producing in situ, hyperpolarized gas. The present invention also provides a device and methods for performing hyperpolarizing gas imaging.

The present invention discloses a system for hyperpolarizing un-polarized gas within an animal, comprising hyper polarization means for hyperpolarizing the un-polarized gas.

It is emphasized that the hyperpolarization of the un-polarized gas is provided in-situ within said animal.

The present invention also discloses a system for hyperpolarizing gas imaging confined within a volume, the volume having a medium therein. The system comprises (a) at least one volume confining an un-polarized gas and at least one medium; and, (b) hyper polarization means for hyperpolarizing the un-polarized gas. It is emphasized that the hyperpolarization of the un-polarized gas is provided within the confined volume.

The present invention also discloses a system for hyperpolarized gas imaging of at least one animal. The system comprises (a) at least one chamber confining a volume in size and shape for accommodating the at least one animal; (b) supplying mechanism for supplying un-polarized gas to the at least one chamber; (c) hyper polarization means for hyperpolarizing the un-polarized gas; and, (d) imaging device for imaging at least a region of the animal. It is emphasized that the hyperpolarization of the un-polarized gas is provided within the confined volume.

It should also be appreciated that the above described description of methods and apparatus are to be interpreted as including apparatus for carrying out the methods, and methods of using the apparatus of any type as well known to a person or ordinary skill, and which need not be described in detail herein for enabling a person of ordinary skill to practice the invention.

Hyperpolarization, according to the present patent, is, inter alia, the selective polarization of nuclear spin in atoms far beyond normal thermal equilibrium. More specifically, it is in the scope of the invention wherein the terms ‘hyperpolarization’ or ‘hyper polarization’ are interchangeably related with the nuclear spin polarization of a material far beyond thermal equilibrium conditions. It is commonly applied to gases such as ¹²⁹Xe and ³He which are then used, for instance, in hyperpolarized magnetic resonance imaging (MRI) of the lungs. Other methods for hyperpolarization include Dynamic Nuclear. Polarisation (DNP) for solid materials at cryogenic temperatures and para-hydrogen used in chemical reactions in liquid solutions (PHIP). DNP of nuclei like ¹³C or ¹⁵N at typically ≈1 K can be coupled with subsequent rapid dissolution yielding a room temperature solution containing hyperpolarized nuclei. This liquid can be used in in vivo metabolic imaging for oncology and other applications. The ¹³C polarization level in the solid is reported as e.g. (64±5)% for a specific setup, and the losses during dissolution and transfer of the sample for actual NMR or MRI measurements can be minimized to a few percent.

The term ‘hyper polarization means’ refers hereinafter to any device, mechanism or system useful for providing hyper polarization. As an example, hyper polarization means is selected in a non-limiting manner from a group consisting laser, ultrasound, RF, microwave, application of heat ans any combination thereof.

The term ‘anesthetic gas’ refers hereinafter to any gas selected from a group consisting of Nitrous oxide (N₂O), Halothane, Enflurane, Isoflurane, Sevoflurane, Desflurane and Xenon, water, oxygen or any combination thereof.

The term ‘magnetic resonance device’ (MRD) applies hereinafter to any Magnetic Resonance Imaging (MRI) device, any Nuclear Magnetic Resonance (NMR) spectroscope, any Electron Spin Resonance (ESR) spectroscope, any Nuclear Quadruple Resonance (NQR) or any combination thereof.

The terms “modality, modalities, mode” refers herein in a non limiting manner to an attribute of the device of the invention which is that the device is provided with more than one means for generating an image or images. In preferred embodiments, the device is provided with NMR means or modalities to generate images of a subject, such as MRI or CT, and also, the very same device is provided with optical means or modalities for generating images of the same subject. Both NMR means and optical means may generate time resolved images.

The term “anatomical imaging” refers hereinafter in a non-limiting manner to NMR based imaging techniques, methods, means and equipment which are used for reconstructing anatomical images, such as Computed Tomography (CT) or Magnetic Resonance (MR) imagers.

The term “functional imaging” refers hereinafter in a non-limiting manner to an optical imaging techniques, methods, means and equipment for detecting or measuring changes in function of an organism, tissue, organ or body part or portion. The functions are, in a non limiting manner, metabolism, blood flow, regional chemical composition, and absorption, as well as any other modality used for molecular imaging. Such functions may be detected by optical detectors or sensors adapted for any technique, method or means selected from a group consisting of optical imaging, optical fluorescence imaging, molecular imaging, bioluminescence, chemiluminescence, fluorescence, UV, IR and/or visible light, Single photon emission computed tomography (SPECT) and Positron emission tomography (PET).

As used herein, the term “subject” refers to any object or living creature inserted in whole or in part into the static magnetic field of a magnetic resonance imaging (MRI) system in order to obtain at least one magnetic resonance image thereof or therefrom.

As used herein, the term “volume of interest” refers to a volume within the subject of which an image is desired. The volume of interest thus may be, for example, the entire subject, an organ within the subject, or a specific volume within an organ within the subject (e.g. the site at which a tumor is suspected to exist).

As used herein, the term “bed” refers to any object, upon a surface of which the subject rests during acquisition of magnetic resonance images by an MRI system. As a non-limiting example, the surface on which the subject rests is the upper surface of the object and is essentially planar. The bed may be translatable to a position located external to the MRI.

As used herein, the term “coil” refers to any generally circular or spiral electrically conducting component, particularly one adapted for use in the transmission or reception of radio-frequency (RF) radiation.

As used herein, the term “midpoint” refers, with reference to a magnetic field, to the point along the magnetic field axis equidistant from two planes perpendicular to the magnetic field axis that together define two limits of a predefined volume.

As used herein, the term “detector” refers to an apparatus adapted for measuring the intensity of a signal impinging upon it and transmitting that intensity to a recording device. The detector will in general include all of the necessary electronics (and, in the case where the signal is made up of photons, optics) to convert the received signal to a current, voltage, or number proportional to the intensity of the signal and means for passing the current, voltage, or number to an appropriate recording device.

As used herein, the term “plurality” refers in a non-limiting manner to any integer equal or greater than 1.

The term ‘about’ applies hereinafter to a measure being ±25% of the defined value.

As described above, the present invention provides means and method of producing in situ, hyperpolarized gas.

More specifically, the present invention also provides a device for performing hyperpolarizing gas imaging.

According to one embodiment of the present invention a system for hyperpolarizing un-polarized gas within an animal is disclosed. The system comprises hyper polarization means for hyperpolarizing the un-polarized gas. It is emphasized that the hyperpolarization of the un-polarized gas is provided in-situ within the animal.

According to another embodiment a system for hyperpolarizing gas imaging confined within a volume is disclosed. The volume having a medium therein.

The system comprises (a) at least one volume confining an un-polarized gas and at least one medium; and, (b) hyper polarization means for hyperpolarizing the un-polarized gas. It is emphasized that the hyperpolarization of the un-polarized gas is provided within the confined volume.

According to another embodiment, the system as defined above, additionally comprising a chamber in fluid communication with the volume, the chamber accommodates at least one animal (selected from mammal, humans, premature babies, reptiles, sea animals, biological specimens, biological organs, mice, rats, rodents, birds, reptiles, amphibians, in vivo biological tissue or organ or ex vivo biological tissue or organ), such that the hyperpolarized gas is supplied from the volume to the chamber.

According to another embodiment of the present invention a system for hyperpolarized gas imaging of at least one animal is provided. The system comprises (a) at least one chamber confining a volume in size and shape for accommodating the at least one animal; (b) supplying mechanism for supplying un-polarized gas to the at least one chamber; (c) hyper polarization means for hyperpolarizing the un-polarized gas; and, (d) imaging device for imaging at least a region of the animal. It is emphasized that the hyperpolarization of the un-polarized gas is provided within the confined volume.

According to another embodiment of the present invention a system for hyperpolarized gas imaging of at least one animal is provided. The system comprises (a) at least one volume confining an un-polarized gas and at least one medium; (b) at least one chamber confining a volume in size and shape for accommodating the at least one animal; the chamber is in fluid communication with the volume; (c) supplying mechanism for supplying un-polarized gas to the at least one volume; (d) hyper polarization means for hyperpolarizing the un-polarized gas; and, (e) imaging device for imaging at least a region of the animal; wherein the hyperpolarization of the un-polarized gas is provided in vitro within the confined volume.

According to another embodiment, the hyperpolarization of the un-polarized gas is paused during the imaging. In other words, once the hyperpolarized gas is inhaled by the animal the imaging takes place and the hyperpolarization is paused.

It is another object of the present invention to provide the systems as defined above, the medium is selected from a group consisting of anesthetic gas, water, oxygen or any combination thereof.

According to another embodiment, the hyper polarization means as described above, is selected from laser, ultrasound, RF, microwave, application of heat or any combination thereof.

According to another embodiment, the gas is selected from helium or Xenon.

According to another embodiment, the imaging device is selected from a group consisting of NMR, MRI, CT, X-ray, ultrasound device, fluorescence device, thermographic device or any combination thereof.

According to another embodiment, the animal is selected from a group consisting of mammal, humans, premature babies, reptiles, sea animals, biological specimens, biological organs, mice, rats, rodents, birds, reptiles, amphibians, in vivo biological tissue or organ or ex vivo biological tissue or organ.

According to another embodiment, the imaging device is selected from a group consisting of NMR, MRI.

The present invention also provides a method for hyperpolarizing un-polarized gas within an animal. The method comprises steps selected from: providing the animal at least partially containing the un-polarized gas; obtaining hyper polarization means for hyperpolarizing the un-polarized gas; hyperpolarizing the un-polarized gas; wherein the step of hyperpolarizing the un-polarized gas is performed in situ within the animal.

According to another embodiment, the method as defined above, additionally comprising step of selecting the hyper polarization means is selected from laser, ultrasound, RF, microwave, application of heat or any combination thereof.

According to another embodiment, the method as defined above, additionally comprising step of selecting the gas is selected from helium or Xenon.

According to another embodiment, the method as defined above, additionally comprising step of pausing the hyperpolarizing of the un-polarized gas during the step of imaging.

According to another embodiment, the method as defined above, additionally comprising step of selecting the animal is selected from a group consisting of mammal, humans, premature babies, reptiles, sea animals, biological specimens, biological organs, mice, rats, rodents, birds, reptiles, amphibians, in vivo biological tissue or organ or ex vivo biological tissue or organ.

The present invention also provides a method for hyperpolarizing un-polarized gas confined within a volume, the volume having a medium therein. The method comprises steps selected inter alia from: providing at least one volume confining an un-polarized gas and at least one medium; obtaining hyper polarization means for hyperpolarizing the un-polarized gas; hyperpolarizing the un-polarized gas; wherein the step of hyperpolarizing the un-polarized gas is performed in vitro within the confined volume.

According to another embodiment, the method as defined above, additionally comprising a chamber in fluid communication with the volume, the chamber accommodates at least one animal (selected from mammal, premature babies, humans, reptiles, sea animals, biological specimens, biological organs, mice, rats, rodents, birds, reptiles, amphibians, in vivo biological tissue or organ or ex vivo biological tissue or organ), such that the hyperpolarized gas is supplied from the volume to the chamber.

According to another embodiment, the method as defined above, additionally comprising step of selecting the hyper polarization means is selected from laser, ultrasound, RF, microwave, application of heat or any combination thereof.

According to another embodiment, the method as defined above, additionally comprising step of selecting the gas is selected from helium or Xenon.

The present invention also provides a method for hyperpolarized gas imaging of at least one animal. The method comprises step selected from: providing at least one chamber confining a volume in size and shape; accommodating the at least one animal within the at least one chamber; supplying un-polarized gas to the at least one chamber; hyperpolarizing the un-polarized gas; and, imaging at least a region of the animal whilst at least one region of the animal contains the hyperpolarized gas for at least a portion of the time required for the imaging. It is emphasized that the step of hyperpolarizing the un-polarized gas is performed within the confined volume.

According to another embodiment, the method as defined above, additionally comprising step of pausing the hyperpolarizing of the un-polarized gas during the step of imaging. In other words, once the hyperpolarized gas is inhaled by the animal the imaging takes place and the hyperpolarization is paused.

According to another embodiment, the method as defined above, additionally comprising step of selecting the imaging device from a group consisting of NMR, MRI, CT, X-ray, ultrasound device, fluorescence device, thermographic device or any combination thereof.

According to another embodiment, the method as defined above, additionally comprising step of selecting the hyperpolarization means from laser, RF, ultrasound, microwave, application of heat or any combination thereof.

According to another embodiment, the method as defined above, additionally comprising step of selecting the animal from a group consisting of mammal, humans, premature babies, reptiles, sea animals, biological specimens, biological organs, mice, rats, rodents, birds, reptiles, amphibians, in vivo biological tissue or organ or ex vivo biological tissue or organ.

According to another embodiment, the method as defined above, additionally comprising step of selecting the gas from helium or Xenon.

The following disclosure is specifically relates to the MRI imaging device.

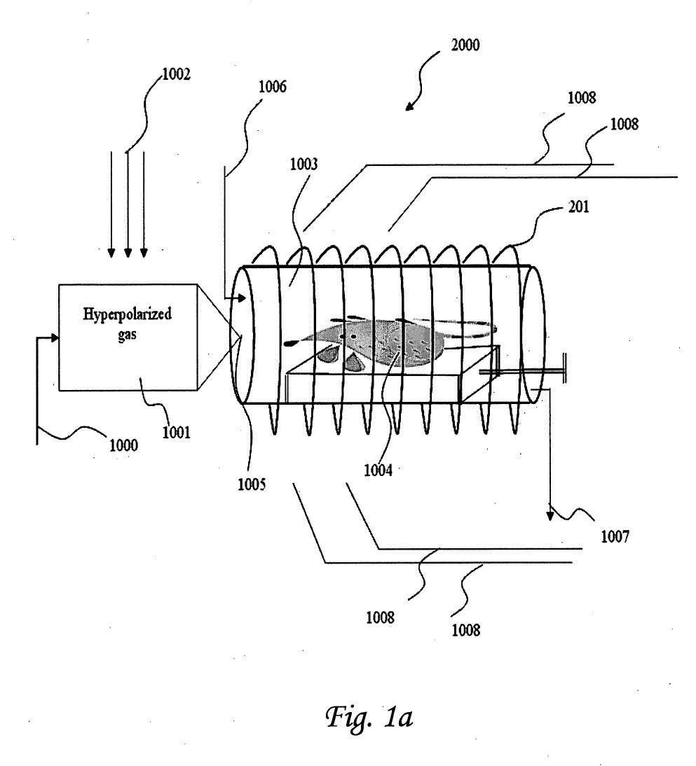

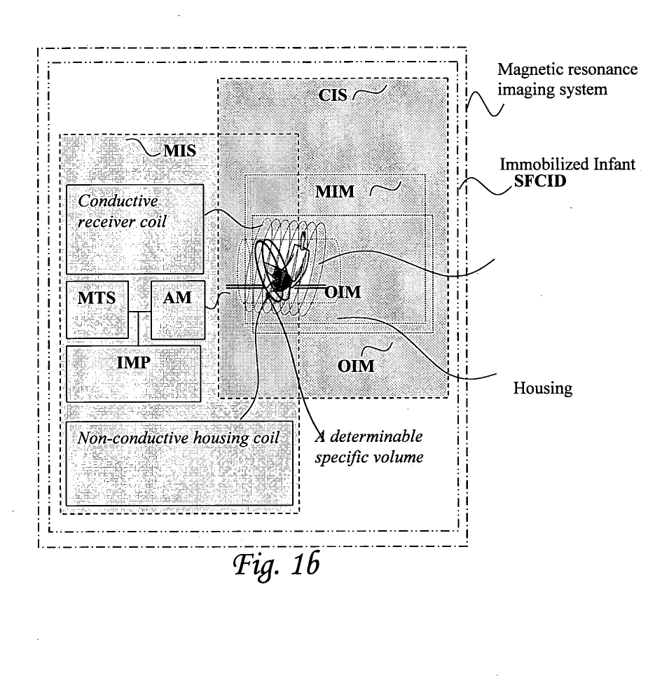

Reference is now made to FIG. 1a, schematically illustrating one embodiment according to the described above.

As can be seen from the figure, the system 2000, comprises supplying means 1000 for supplying un-polarized gas into volume 1001 confining the un-polarized gas.

The system also comprises hyper polarization means 1002 for hyperpolarizing the un-polarized gas in situ within the volume 1001.

The Volume 1001 is coupled by means of valve 1005 to chamber 1003 which accommodates animal 1004.

Supplying means 1006 are adapted to supply anesthetic gas to the chamber.

Draining means 1007 are also coupled to the chamber, adapted to drain the anesthetic gas.

Numerical reference 201 is a transmit coil adapted to produce pulses of RF radiation.

The system may additionally comprise fiber optics 1008 for the imaging.

It should be emphasized that Volume 1001 and chamber 1003 can be united (i.e., the same chamber).

Reference is now made to FIG. 1b, schematically illustrating in a non-limited manner a block diagram of a magnetic resonance imaging system according to one embodiment of the invention. The magnetic resonance imaging system comprises a novel spatially fixed coupled imaging device (SFCID) useful for producing combined anatomical and real time functional light images. The SFCID functionally incorporates a maneuverable imaging system MIS with a coupled imaging system CIS. The maneuverable imaging system (MIS) contains, inter alia, an imaging platform (IMP) accommodating an immobilized subject positioned within a nonconductive housing. The IMP is contained within a radio frequency coil system (RFCS) for imaging one or more regions of a subject. The RFCS is adapted either to reversibly translate (i) at least one conductive receiver coil, and/or (ii) at least a portion of the IMP, in at least one nonconductive housing coil to at least one fixed position to an accuracy of not less than about 3 mm, while the subject remains within the MIS. The RFCS includes, inter alia, a mechanical translation system (MTS) adapted for providing linear motion to the immobilized subject and for reproducibly fixing the position of the immobilized subject to within a range of about 3 to about 60 mm. The RFCS also includes attaching means (AM) for connecting the housing to the MTS. The coupled imaging system (CIS) is adapted to image at least one specific region of the immobilized subject, and to integrate (i) at least one MRD imaging module (MIM) configured for providing three dimensional anatomical images; with (ii) at least one optical imaging module (OIM), coupled to the IMP and configured for detecting photons emitted or reflected by the region of the immobilized subject so as to generate real time functional light images of a functionally active part of the region of the immobilized subject. Thus, the functional incorporation of coupled MIM and OIM in the IMP provides one or more multi-modular fused, real-time images of the region of the immobilized subject located within a determinable specific volume.

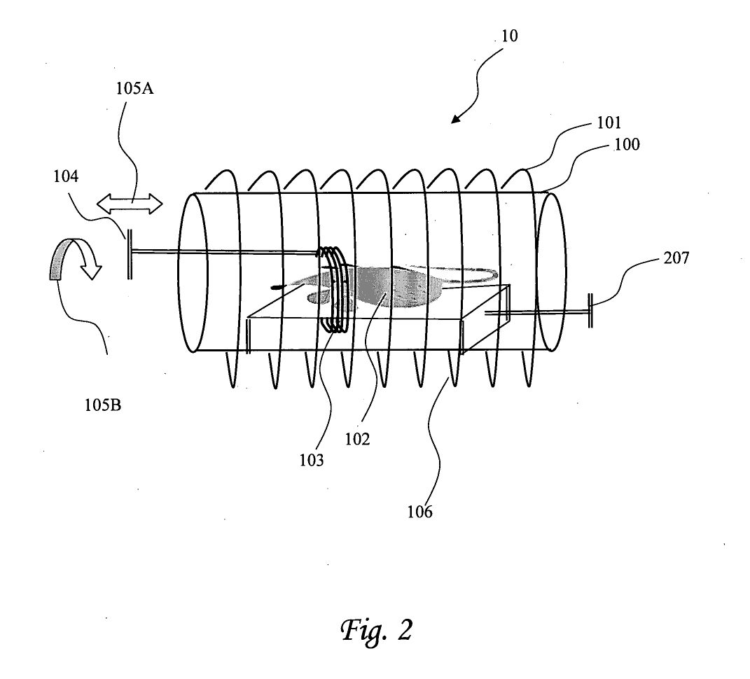

Reference is now made to FIG. 2, which presents a schematic drawing (side view) of an SFCID 10 according to yet another embodiment of the invention, that includes the receiver coil assembly disclosed in the present invention. A static magnetic field is created by a magnet (not shown) external to MRI chamber 100. The magnet may be a superconducting magnet or a permanent magnet of any appropriate geometrical design. Also not shown in FIG. 2 are gradient coils that produce appropriate gradient magnetic fields. The design and construction of such magnets and coils is well-known in the art. A transmit coil 101, located external to MRI chamber 100, provides RF pulses to excite magnetic nuclei within the static magnetic field according to principles well-known in the art. Subject 102 (here e.g., a mouse) is positioned within chamber 100 such that the volume of interest is located within the static magnetic field and within the volume enclosed by transmit coil 101; in another embodiment, subject 102 is a human being, and the MRI instrument is adapted to obtain images of the whole body. In alternative embodiments, only a part of the subject's body (e.g. the head or a limb) is located within chamber 100; in further alternative embodiments, the subject is not a human being (as a non-limiting example, the subject can be a small mammal such as a rat or rabbit; in general, in these embodiments, the entire animal is located within the chamber). In the embodiment shown, subject 102 lies on bed 106 or similar furniture. It is yet in the scope of the invention wherein (i) both coils 101 and 103 are within the internal portion of housing 100, or (ii) wherein, as shown, coil 101 located externally to the housing and coil 103 located internally, within the housing.

Receiver coil 103 substantially encircles the volume of interest, and thus may be designed, for example, to encircle the entire body of the subject, or a limb or body part thereof, depending on the specific location within the subject of the volume of interest. Receiver coil 103 is positioned so it is a close as possible to the volume of interest. The receiver coil may be any type of RF coil, e.g. a solenoid, a Helmholtz coil, or a surface coil (loop). The inner coil does not have to be homogeneous. In the embodiment shown in FIG. 1, there is a single receiver coil; in alternative embodiments, a plurality of independent coils is present. Receiver coil is attached to mechanical translation device 104.

The mechanical translation device is adapted to move the receive coil to any predetermined position along the axis defined by the static magnetic field and in rotation around the axis (see arrows 105A and 105B, respectively). The mechanical translation device can use any appropriate means known in the art for moving the receiver coil that is also adapted for fixing its position to within X mm (e.g. via a stepper motor); X is any integer number, e.g., X is ranging between about 0.1 mm to about 50 mm; between about 5 mm to about 500 mm, between about 50 mm to 1.5 m etc. Once the receiver coil is properly positioned, MRI can proceed according to any appropriate pulse/detection scheme.

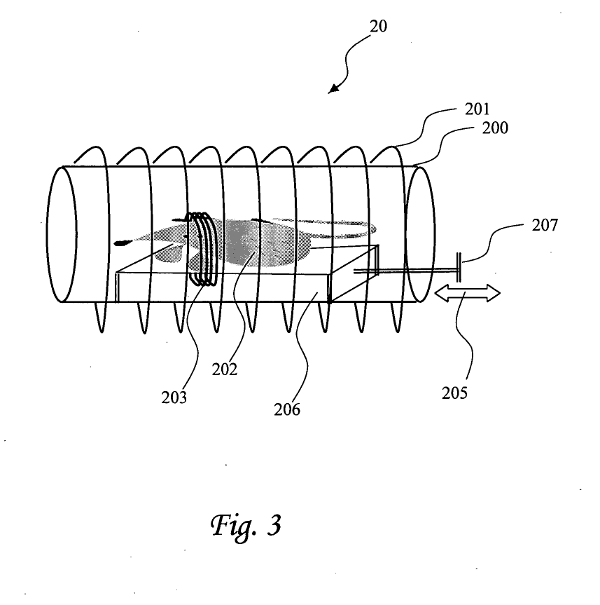

Reference is now made to FIG. 3, which illustrates schematically a side view of another embodiment 20 of the SFCID herein disclosed. This embodiment comprises all of the features of the previous embodiment: an MRI-fitted chamber 200 into which subject 202 or a portion thereof is introduced; a transmit coil 201 adapted to produce pulses of RF radiation; at least one receiver coil 203 that substantially encircles the volume of interest; means 204 for moving the receiver coil or coils in the direction of arrows 205 (i.e. parallel to the magnetic field axis of the static magnetic field); and a bed 206 upon which the subject is placed. As with the previous embodiment, the magnet that produces a static magnetic field, the gradient coils that produce magnetic field gradients, the associated electronics and controllers, all of which are well-known in the art, are not shown. This embodiment contains in addition mechanical means 207 for translating bed 206 along the direction indicated by arrows 205. This mechanical means may be any means known in the art for moving the bed to a desired location. The motion of the bed may be independent of mechanical means 204 that are used to translate receiver coil 203, or the two mechanical translation devices may be coupled so that, as non-limiting examples, the bed and the receiver coil move in tandem; they may be coupled to move in opposite directions; or they may be coupled so that motion of one is set to a predetermined fraction of the other (e.g. moving the bed through a distance D moves the coil through a distance 0.1 D in a predetermined direction relative to the direction of motion of the bed). In this embodiment, it is possible to move the subject so that the volume of interest is located at the midpoint of the static magnetic field and then to scan the receiver coil over the subject such that the volume of interest is imaged. This embodiment also enables fixing the receiver coil at the midpoint of the static magnetic field and moving the subject through the coil at a predetermined velocity so that the volume of interest is scanned with the coil remaining stationary at the point at which its spatial resolution is highest.

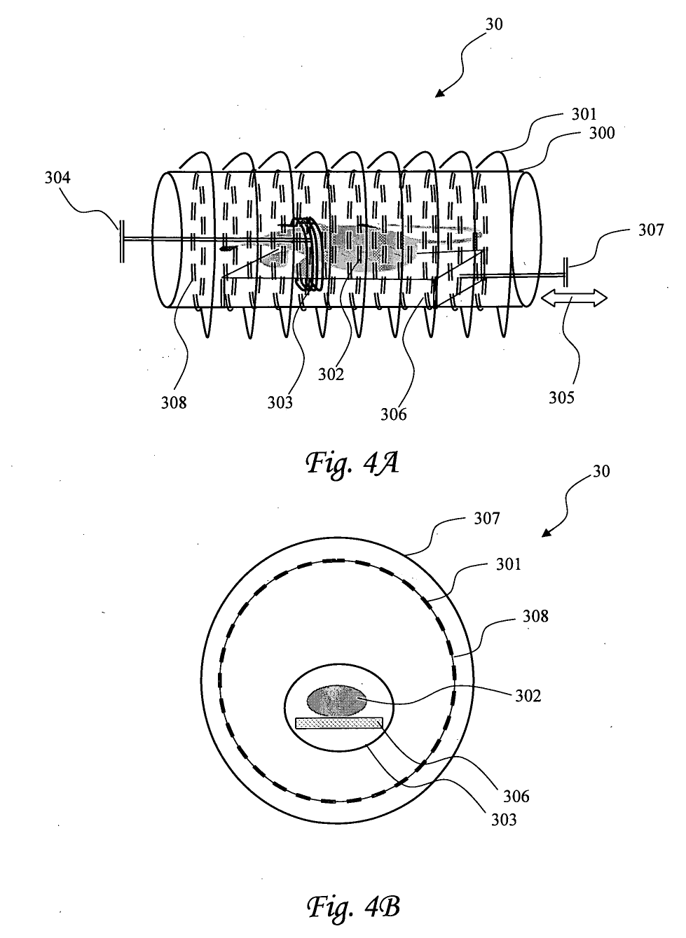

Reference is now made to FIG. 4, showing schematically a side view of a third embodiment 30 of the SFCID herein disclosed. In addition to the elements recited in the previous embodiment (components 300-307 of embodiment 30 are exactly analogous to components 200-207 of embodiment 20), this embodiment contains a plurality of N detectors 308 disposed about the circumference of chamber 300; N is any integer number, e.g. and in a non-limiting manner, N ranges between about 1 to about 20, between about 3 to about 300 or between about 30 to about 3000. The general disposition of these detectors is shown in FIG. 4a; in various embodiments, the detectors may be disposed along the entire length of the chamber, or only along a predetermined fraction of the length of the chamber, according to the needs of the particular imaging data needed. It is in the scope of the invention wherein detectors 308 are selected, in a non-limiting manner, from a group consisting of bioluminescence, chemiluminescence, fluorescence, UV, IR and/or visible light and any combination hereof. According to the specific embodiment of the present invention, spatial location of the optical detectors is provided, and hence, triangulation of the imaged data is possible.

A cross-sectional slice (front or rear view) of a typical embodiment is shown schematically in FIG. 4b; in this embodiment, the detectors are disposed within the wall of the chamber. In alternative embodiments, the detectors may be attached to the inside of the chamber either in addition to or in place of detectors disposed within the wall of the chamber.

The detectors are adapted for at least one additional kind of imaging in addition to MRI; non-limiting examples include SPECT, PET, and fluorescence. The detectors are connected by any appropriate means as known in the art to a recording device (e.g., in the case of fluorescence, the detectors may be connected via appropriate fiber-optic cables to a CCD/computer assembly) such that the signal measured by each detector is separately recorded and stored. In this embodiment, the plurality of detectors enables collection and calculation of truly 3-dimensional (3D) information. In addition, the presence of the mechanical translation means 304 and 307 for moving the coil and/or bed enables direct line-of-sight access from the subject to the detectors during the collection of the image by the additional imaging means, i.e., the receiver coil is moved out of the way during the collection of the subsequent image or images without moving the subject from its position during the collection of the MRI data.

According to the embodiments defined and illustrated above, and since the subject remains stationary during the entire data collection procedure, superposition of the images obtained by MRI and by the additional method or methods is straightforward, and enables true 3-dimensional imaging of the subject.

It is thus according to yet another embodiment of the invention, wherein the functional imager disclosed in the present invention is an optical imaging modality, and the detector is an optical detector. For multi dimensional imaging, usually a plurality of detectors is required. The detectors can transform the acquired data either by optic fibers, compatible with the imaging modalities used, or by any other means of transforming information. The subject handling system can also serve to adjust the desired location of the subject in relation to the imaging device and/or strap the subject to avoid movement during the acquisition process. The device can additionally further comprise sensors to regulate the subjects or the environments conditions inside the multimodality imaging device.

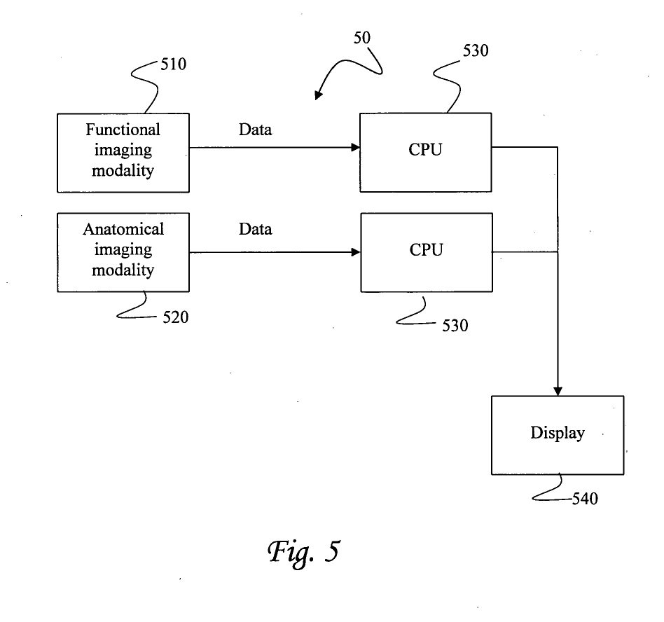

According to yet another embodiment of the invention, as set forth in a schematic manner in block diagram of FIG. 5, a multimodality imaging system is disclosed. The system (50) comprises of a functional imager (510) and an anatomical imaging modality (520), which transform data into processors (see e.g., CPUs 530). Since the location of the subject remains the same during both scans, the reconstructed images can be fused into a single image, displaying the correlation between function and anatomy. The fused image can then be saved or displayed by means of displayer 540 in any required form (either hard copy or soft copy).

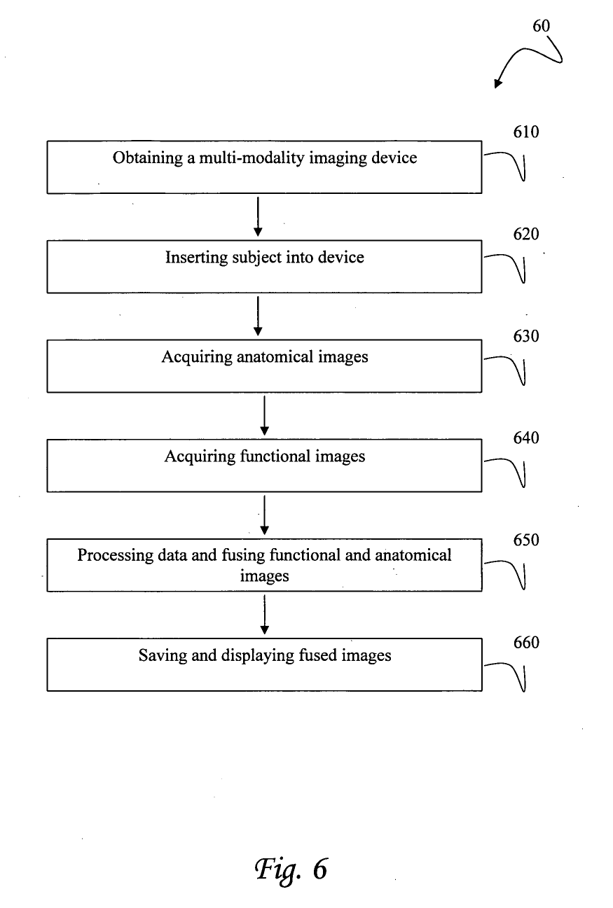

Reference is now made to FIG. 6, schematically shows a flow chart according to one embodiment of the invention, displaying in a non-limiting manner a method of acquiring in vivo fused images using a multimodality imaging device. The method comprises, inter alia, steps of obtaining a multi-modality spatially fixed coupled imaging device (SFCID) 610, inserting a subject into the device 620, acquiring anatomical images 630, acquiring functional images 640, processing data and fusing functional and anatomical images 650, and saving and displaying fused images 660.

In an illustrative example which is provided below in a non-limiting manner, an immobilized subject of study is inserted into the SFCID as defined in the present invention, both anatomical NMR images are acquired and functional images are acquired. The data is processed and fusing of functional and anatomical images is carried out. The fused images are saved and displayed. The functional images, which have been generated by the optical data from the optical sensor array in a preferred embodiment, represent e.g., aspects of the metabolic activity of the tumor. Since the SFCID provides time resolved images, the metabolism of a tumor is monitored over time. This is very important for a wide range of studies, such as cell uptake studies, as well as diagnostic studies of the progress of a malignancy or proliferative cell or tissue disorder. Different drugs can be administered in vivo to a subject undergoing tumor studies or treatment, and the effect on the metabolically active or functionally active part of the tumor can be observed through time. Many malignant tumors have functionally active areas and less active or dead areas. These areas can be monitored accurately in time, in three dimensions.

Since the functional images of the present invention are provided as real time acquisitions, they can be displayed on a single anatomical image which was taken prior to the functional image, in which case the reconstructed fused image will vary with time for each anatomical slice section.

Some anatomical imaging modalities are also capable of producing real-time anatomical images, for example, perfusion images in either CT or MRI, MR-Echo sequences etc. Furthermore, images are sometimes gated, either according to the cardiac rhythm or to the respiratory rhythm. In both cases, both functional and anatomical images can either be acquired simultaneously, or be acquired at different times, and optionally be correlated according to the gating. It is also possible for the functional image to be acquired prior to the acquisition of the anatomical image, or for both modalities to work alternately in the course of one session.

According to one embodiment of the invention, the magnetic resonance imaging (MRI) system includes a detached receiver coil that has the following characteristics: (1) it comprises a single receiver coil independent of the transmit coil; (2) the receiver coil is positionable to allow scanning of a particular volume of choice; (3) the instrument is designed to allow the volume of interest and the receiver coil to be placed at the midpoint of the static magnetic field; (4) the system is adapted not only for acquisition of a 3D MRI image with high sensitivity, positional accuracy, and SNR, but also for acquisition of a 3D image obtained by at least one other spectroscopic method without moving the body being imaged and without the receiver coil blocking the signal being detected by the other method or methods.

According to another embodiment of the invention, the MRI system comprises an RF coil system for imaging one or more regions of a subject. The RF system comprises, inter alia, (a) a coil comprising at least one conductive coil in at least one nonconductive housing; (b) a mechanical translation system adapted for providing linear motion to an attached object and for reproducibly fixing the position of the attached object to within distance X; and (c) attaching means for connecting the housing to the mechanical translation system. The coil system is adapted reversibly to translate the coil to at least one fixed position to an accuracy of about X mm while the subject remains within the magnetic resonance imaging system. X mm (e.g. via a stepper motor); X is any integer number, e.g., X is ranging between about 0.1 mm to about 50 mm; between about 5 mm to about 500 mm, between about 50 mm to 1.5 m etc.

According to another embodiment of the invention, wherein the coil is chosen from the group consisting of (a) a solenoid, (b) a Helmholtz coil, and (c) a surface coil.

According to another embodiment of the invention, the MRI system comprises, inter alia, (a) a magnet for generating a stable magnetic field in a volume, the stable magnetic field defining a magnetic field axis; (b) a plurality of coils for establishing at least one magnetic gradient within the volume; (c) at least one coil for applying pulses of RF radiation to excite nuclear spins of a body located within the volume; and (d) at least one receiver coil as described above, the at least one receiver coil adapted to optimize reception of resonance signals emanating from the body. The magnetic resonance imaging system is adapted to provide at least one magnetic resonance image of at least one predetermined volume within the subject.

According to another embodiment of the invention, least one of the fixed positions is located outside of the volume.

According to another embodiment of the invention, in the MRI system, one of the fixed positions is the point at which the optimized reception occurs at the point along the midpoint of the stable magnetic field along the magnetic field axis.

According to another embodiment of the invention, in the MRI system, at least one of the fixed positions is located outside of the volume and one of the fixed positions is the point at which the optimized reception occurs at the point along the midpoint of the stable magnetic field along the magnetic field axis

According to another embodiment of the invention, the MRI system comprises, inter alia, (a) a second mechanical translation system adapted for providing linear motion to an attached object and for reproducibly fixing the position of the attached object to within about X mm; and (b) attaching means for connecting the bed to the mechanical translation system. It is within the essence of the invention wherein the bed is adapted reversibly to translate relative to the volume independent of the translation of the RF coil.

According to another embodiment of the invention, the MRI system comprises, inter alia, coupling means for connecting the mechanical translation system attached to the bed with the mechanical translation system attached to the RF coil, wherein the motions of the bed and the coil are interdependent.

According to another embodiment of the invention, the MRI system comprises, inter alia, (a) a plurality of detectors disposed about the perimeter of the volume; and (b) means for transmitting a signal from each of the plurality of detectors to a controller located external to the volume. It is within the essence of the invention wherein the magnetic resonance imaging system is adapted for performing at least one type of imaging method in addition to magnetic resonance imaging.

According to another embodiment of the invention, a method for magnetic resonance imaging of a volume of interest in an object to be examined is provided by means of a moveable RF coil system. The method comprises, inter alia, steps of: (a) introducing the object to a predetermined position within a stable magnetic field generated by a magnet; (b) placing a positionable RF receiver coil in proximity to the object such that the position of the RF receiver coil is fixed to within X mm and such that at least part of the volume of interest is located within the volume defined by the coil; (c) exciting nuclear magnetization in the volume of interest by applying RF pulses and magnetic field gradients according to a predetermined imaging protocol; (d) receiving RF imaging signals generated in the RF receiver coil by the excited nuclear magnetization; and (e) reconstructing a magnetic resonance image of the volume of interest from the received magnetic resonance imaging signals and from the position of the RF receiver coil. The method yields an accurate three-dimensional magnetic resonance image of the volume of interest.

According to another embodiment of the invention, the method is provided by a means of an RF receiver coil, which is adapted to optimize reception of resonance signals emanating from the volume of interest, and further wherein the step of placing a positionable RF receiver coil in proximity to the object further includes the step of placing the positionable RF receiver at the point along the midpoint of the stable magnetic field along the magnetic field axis.

According to another embodiment of the invention, the aforesaid method is provided by steps of introducing the object to a predetermined position within a stable magnetic field generated by a magnet and of placing a positionable RF receiver coil in proximity to the object are performed by mechanical means adapted to allow independent motion of the body and of the RF receiver coil.

According to another embodiment of the invention, a method for magnetic resonance imaging of a volume of interest in an object to be examined by means of a moveable RF coil system and at least one other imaging technique of the volume of interest is provided. The method comprises, inter alia, steps of: (a) introducing the object to a predetermined position, the predetermined position located within a volume at least part of the interior of which contains stable magnetic field generated by a magnet and about the perimeter of which a plurality of detectors are disposed; (b) placing a positionable RF receiver coil in proximity to the object such that the position of the RF receiver coil is fixed to within X mm and such that at least part of the volume of interest is located within the volume defined by the coil; (c) exciting nuclear magnetization in the volume of interest by applying RF pulses and magnetic field gradients according to a predetermined imaging protocol; (d) receiving RF imaging signals generated in the RF receiver coil by the excited nuclear magnetization; (e) reconstructing a magnetic resonance image of the volume of interest from the received magnetic resonance imaging signals and from the position of the RF receiver coil; and (f) transmitting a signal from each of at least one of the plurality of detectors to a controller located external to the volume, the transmission commencing at a predetermined time relative to the commencement of step (c) and continuing for a predetermined length of time.

According to another embodiment of the invention, the aforesaid method is provided wherein the at least one other imaging technique is chosen from the group consisting of (a) fluorescence spectroscopy; (b) SPECT; (c) PET; (d) any combination of the above.

1-87. (canceled)

88. A system for hyperpolarizing un-polarized gas within an animal, comprising hyper polarization means for hyperpolarizing said un-polarized gas; wherein said hyperpolarization of said un-polarized gas is provided in-situ within said animal.

89. The system according to claim 88, wherein at least one of the following is being held true (a) said hyper polarization means is selected from laser, ultrasound, RF, microwave, application of heat or any combination thereof; (b) said gas is selected from helium or xenon; (c) said animal is selected from a group consisting of mammal, humans, premature babies, reptiles, sea animals, biological specimens, biological organs, mice, rats, rodents, birds, reptiles, amphibians, in vivo biological tissue or organ or ex vivo biological tissue or organ; and any combination thereof.

90. A system for hyperpolarizing un-polarized gas confined within a volume, said volume having a medium therein, comprising
a. at least one volume confining an un-polarized gas and at least one medium; and,
b. hyper polarization means for hyperpolarizing said un-polarized gas;
wherein said hyperpolarization of said un-polarized gas is provided in vitro within said confined volume.

91. The system according to claim 90, wherein at least one of the following is being held true (a) said hyper polarization means is selected from laser, ultrasound, RF, microwave, application of heat or any combination thereof; (b) said gas is selected from helium or Xenon; (c) said animal is selected from a group consisting of mammal, humans, premature babies, reptiles, sea animals, biological specimens, biological organs, mice, rats, rodents, birds, reptiles, amphibians, in vivo biological tissue or organ or ex vivo biological tissue or organ; (d) said system additionally comprising a chamber in fluid communication with said volume; said chamber accommodates at least one animal, such that said hyperpolarized gas is supplied from said volume to said chamber; (e) said medium is selected from a group consisting of anesthetic gas, water, oxygen or any combination thereof; and any combination thereof.

92. A system for hyperpolarized gas imaging of at least one animal, comprising:
a. at least one volume confining an un-polarized gas and at least one medium;
b. at least one chamber confining a volume in size and shape for accommodating said at least one animal; said chamber is in fluid communication with said volume;
c. supplying mechanism for supplying un-polarized gas to said at least one volume;
d. hyper polarization means for hyperpolarizing said un-polarized gas; and,
e. imaging device for imaging at least a region of said animal;
wherein said hyperpolarization of said un-polarized gas is provided in vitro within said confined volume.

93. A system for hyperpolarized gas imaging of at least one animal, comprising:
a. at least one chamber confining a volume in size and shape for accommodating said at least one animal;
b. supplying mechanism for supplying un-polarized gas to said at least one chamber;
c. hyper polarization means for hyperpolarizing said un-polarized gas; and,
d. imaging device for imaging at least a region of said animal;
wherein said hyperpolarization of said un-polarized gas is provided in vitro within said confined volume.

94. The system according to claim 92, wherein at least one of the following is being held true (a) said medium is selected from a group consisting of anesthetic gas, water, oxygen or any combination thereof; (b) anesthetic gas, water, oxygen or any combination thereof is supplied to said chamber; (c) said imaging device is selected from a group consisting of NMR, MRI, CT, X-ray, ultrasound device, fluorescence device, thermographic device or any combination thereof; (d) said hyper polarization means is selected from laser, ultrasound, microwave, RF, application of heat or any combination thereof; (e) said gas is selected from helium or Xenon; (f) said animal is selected from a group consisting of mammal, humans, premature babies, reptiles, sea animals, biological specimens, biological organs, mice, rats, rodents, birds, reptiles, amphibians, in vivo biological tissue or organ or ex vivo biological tissue or organ.

95. The system according to claim 94, wherein said NMR/MRI system comprising a spatially fixed coupled imaging device (SFCID) for producing combined anatomical and real time functional light images, the SFCID functionally incorporates a maneuverable imaging system MIS with a coupled imaging system CIS:
a. said maneuverable imaging system (MIS) contains an imaging platform (IMP) accommodating an immobilized subject positioned within a nonconductive housing; said IMP is contained within a radio frequency coil system (RFCS) for imaging one or more regions of a subject; said RFCS is adapted either to reversibly translate (i) at least one conductive receiver coil, and/or (ii) at least a portion of said IMP, in at least one nonconductive housing coil to at least one fixed position to an accuracy of not less than about 3 mm while said subject remains within said MIS; said RFCS includes: a mechanical translation system (MTS) adapted for providing linear motion to said immobilized subject and for reproducibly fixing the position of said immobilized subject to within a range of about 3 to about 60 mm; and, attaching means (AM) for connecting said housing to said MTS; and,
b. said coupled imaging system (CIS) adapted to image at least one specific region of said immobilized subject, and to integrate (i) at least one MRD imaging module (MIM) configured for providing three dimensional anatomical images; with (ii) at least one optical imaging module (OIM), coupled to said IMP and configured for detecting photons emitted or reflected by said region of said immobilized subject so as to generate real time functional light images of a functionally active part of said region of said immobilized subject; the functional incorporation of coupled MIM and OIM in said IMP provides one or more multi-modular fused, real-time images of said region of said immobilized subject located within a determinable specific volume.

96. The system according to claim 95, wherein at least one of the following is being held true (a) said RF coil is selected from the group consisting of a solenoid, a Helmholtz coil, and a surface coil; (b) at least one of said fixed positions is located outside of said nonconductive housing; (c) the imaging platform (IMP) is a bad; and any combination thereof.

97. The system according to claim 95, further comprising:
a. a nonconductive housing which defines a volume of interest (VOI);
b. a magnet adapted for generating a stable magnetic field with a defined magnetic field axis in said VOI;
c. a plurality of coils adapted for establishing at least one magnetic gradient within the VOI;
d. at least one non-conductive housing coil (NCHC) adapted for applying pulses of RF radiation to excite nuclear spins within the immobilized subject in said VOI; and,
e. at least one conductive receiver coil (CRC) located within said NCHC;
wherein said CRC is adapted to optimize reception of resonance signals emanating from said immobilized subject within a determinable specific volume provided within said VOI.

98. The system according to claim 95, wherein one of said fixed positions is the point at which said optimized reception occurs at the point along the midpoint of said stable magnetic field along said magnetic field axis; further wherein at least one of said fixed positions is located outside of said volume and one of said fixed positions is the point at which said optimized reception occurs at the point along the midpoint of said stable magnetic field along said magnetic field axis.

99. The system of claim 95, further comprising:
a. a second mechanical translation system (MTS) adapted for providing linear motion to said immobilized subject and for reproducibly fixing the position of said immobilized subject within a range of about 3 mm to about 60 mm; and,
b. attaching means (AM) for connecting said IMP or portions thereof to said MTS;
wherein said IMP is adapted reversibly to translate relative to said determinable specific volume independent of said translation of said CRC; further wherein said AM adapted to connect said mechanical translation system (MTS) attached to said IMP with said MTS attached to said CRC, and further wherein the motions of said IMP and CRC are interdependent.

100. The system of claim 95, wherein said optical imaging module (OIM) comprises
a. a plurality of detectors functionally incorporated within the perimeter of said housing; and,
b. means for transmitting a signal from each of said plurality of detectors to a controller located external to said volume;
wherein said functional incorporation of said plurality of detectors within said hosing enables production combined anatomical and real time functional light images.

101. The system of claim 95, wherein said optical imaging module (OIM) comprises
a. a plurality of optic fibers functionally incorporated within the perimeter of said housing; and,
b. means for transmitting a signal from each of said plurality of optic fibers to a controller located external to said volume;
wherein said functional incorporation of said plurality of optic fibers within said hosing enables production combined anatomical and real time functional light images.

102. The system of claim 100, wherein the coupled imaging system (CIS) provides an imaging method selected from the group consisting of (a) fluorescence spectroscopy, (b) SPECT, (c) PET, and any combination of the above; and further wherein said plurality of either detectors and/or optics fibers are adapted for detecting signals typical of said at least one additional imaging method.

103. The system of claim 95, wherein at least one of the following is being held true (a) said spatially fixed coupled imaging device (SFCID) is adapted for 3-dimensional (3D) multimodal imaging; (b) said device is provided with a self-fastening cage of a magnetic resonance device (MRD) (100) for providing a homogeneous, stable and uniform magnetic field therein, characterized by an outside shell comprising at least three flexi-jointed superimposed walls (1) disposed in a predetermined arrangement clockwise or counterclockwise; (c) said immobilized subject is either (a) a small mammal; (b) said immobilized subject is selected from a group consisting of humans, premature babies, mammals, biological specimens, biological organs, mice, rats, rodents, birds, reptiles, amphibians, in vivo biological tissue or organ or ex vivo biological tissue or organ; and any combination thereof.

104. The system of claim 95, wherein said MRD comprises:
a. at least six side-magnets arranged in two equal groups being in a face-to-face orientation in a magnetic connection with the cage walls characterized by an outside shell comprising at least three flexi-jointed superimposed walls disposed in said same predetermined arrangement of the cage walls, increasing the overall strength of the magnetic field provided in said cage;
b. at least two pole-magnet pieces, arranged in a face-to-face orientation in between said side-magnets; and,
c. at least two main-magnets, located on said pole-pieces, arranged in a face-to-face orientation, generating the static magnetic field therein said cage.

105. The system of claim 95, comprising at least one of the following is being held true (a) said Central Processing Unit (CPU) for processing and integrating said three dimensional MRD images received from said at least one MRD imaging module (MIM) and said real time functional light images received from said at least one optical imaging module (OIM); (b) said CPU is provided with means to display said three dimensional MRD images and said real time light images; (c) said CPU is provided with means for distinguishing said real time light images from said three dimensional NMR images of said region of said immobilized subject such that functionally active parts of said region of said immobilized subject are identifiable in real time; and any combination thereof.

106. The system of claim 95, wherein said MRD module comprises at least one selected from a group consisting of (a) Two-Dimensional Fourier Transform (2DFT) means and slice selection means for building said image; (b) CT means; (c) MRI means; (d) Three-Dimensional Fourier Transform (3DFT) means for building said image; (e) projection reconstruction means for building said image; (f) point by point image building means for building said image; (g) line by line image building means for building said image; (h) static field gradient image building means for building said image; (i) RF field gradient image building means for building said image; and any combination thereof.

107. The system of claim 95, wherein said optical imaging module comprises at least one selected from a group consisting of (a) light detector array including a plurality of light detectors distributed around said imaging platform in a predetermined manner for providing three dimensional real time light images of said region said immobilized subject; (b) said optical imaging module is provided with means for detecting bioluminescence of said region of said immobilized subject; (c) means for detecting chemiluminescence of said region of said immobilized subject; (d) means for detecting fluorescence of said region of said immobilized subject; (e) means for detecting near infra-red fluorescence of said region of said immobilized subject; (f) means for single photon emission computed tomographic imaging (SPECT) of said region said immobilized subject; (g) means for Positron emission tomographic imaging (PET) of said region of said immobilized subject; (h) photon counting sensitivity means; (i) means for selectively detecting excitation pulses traveling back from said region of said immobilized subject; (j) means for synchronizing said excitation pulses.

108. A method for hyperpolarized gas imaging of at least one animal, said method comprising steps of:
a. providing at least one chamber confining a volume in size and shape;
b. accommodating said at least one animal within said at least one chamber;
c. supplying un-polarized gas to said at least one chamber;
d. hyperpolarizing said un-polarized gas; and,
e. imaging at least a region of said animal whilst at least one region of said animal contains said hyperpolarized gas for at least a portion of the time required for said imaging;
f. wherein said step of hyperpolarizing said un-polarized gas is performed within said confined volume.

109. The method according to claim 108, additionally comprising at least one step selectee from a group consisting of (a) selecting said imaging device from a group consisting of NMR, MRI, CT, X-ray, ultrasound device, fluorescence device, thermographic device or any combination thereof; (b) selecting said hyperpolarization means from laser, RF, ultrasound, microwave, application of heat or any combination thereof; (c) selecting said animal from a group consisting of mammal, humans, premature babies, reptiles, sea animals, biological specimens, biological organs, mice, rats, rodents, birds, reptiles, amphibians, in vivo biological tissue or organ or ex vivo biological tissue or organ; (d) selecting said gas from helium or Xenon; and any combination thereof.

110. The method according to claim 108, additionally comprising at least one step selected from a group consisting of (a) pausing said hyperpolarizing of said un-polarized gas during said step of imaging; (b) producing combined anatomical and real time functional light images, by functionally incorporating a maneuverable imaging system MIS with a coupled imaging system CIS.

111. The method according to claim 110, wherein said step of producing additionally comprising steps of:
a. providing a spatially fixed coupled imaging device (SFCID) in a magnetic resonance imaging system, providing said MIS with an imaging platform (IMP) accommodating an immobilized subject positioned within a nonconductive housing;
b. providing said IMP within a radio frequency coil system (RFCS) for imaging one or more regions of a subject;
c. providing said RFCS with means to either reversibly translate (i) at least one conductive receiver coil (CRC), and/or (ii) at least a portion of said IMP, in at least one nonconductive housing coil (NCHC) to at least one fixed position to an accuracy of not less than about 3 mm while said subject remains within said MIS;
d. further providing said RFCS with a mechanical translation system (MTS), and attaching means (AM) for connecting said housing to said MTS by means of said MTS, maneuvering said immobilized subject in a linear motion, and reproducibly fixing the position of said immobilized subject to within a range of about 3 to about 60 mm;
e. imaging at least one specific region of said immobilized subject, by integrating (i) at least one MRD imaging module (MIM) configured for providing three dimensional anatomical images; with (ii) at least one optical imaging module (OIM), coupled to said IMP and configured for detecting photons emitted or reflected by said region of said immobilized subject thus generating real time functional light images of a functionally active part of said region of said immobilized subject; and,
f. functionally incorporating MIM and OIM in said IMP, thus providing one or more multi-modular fused, real-time images of said region of said immobilized subject located within a determinable specific volume; wherein said method is useful for optimizing reception of resonance signals emanating from said determinable specific volume, wherein said step of placing an NCHC in proximity to said object further includes a step of placing said NCHC at the point along the midpoint of said stable magnetic field along said magnetic field axis.

112. The method of claim 111, comprising the steps of:
a. introducing said immobilized subject to a determinable specific position within a stable magnetic field generated by a magnet;
b. placing a positionable NCHC in proximity to said immobilized subject such that the position of said NCHC is fixed to within about 3 mm to about 60 mm and such that at least part of said volume of interest is located within the volume defined by said NCHC;
c. exciting nuclear magnetization in the volume of interest by applying RF pulses and magnetic field gradients according to a predetermined imaging protocol;
d. receiving RF imaging signals generated in said NCHC by said excited nuclear magnetization; and,
e. reconstructing a magnetic resonance image of said determinable specific volume from the received magnetic resonance imaging signals and from said position of said NCHC.

113. The method of claim 111, comprising:
a. introducing said immobilized subject to a determinable specific position, said position located within a volume at least part of the interior of which contains stable magnetic field generated by a magnet and about the perimeter of which a plurality of detectors are disposed;
b. placing a positionable RF receiver coil in proximity to said object such that the position of said RF receiver coil is fixed to within X mm and such that at least part of said volume of interest is located within the volume defined by said coil;
c. exciting nuclear magnetization in the volume of interest by applying RF pulses and magnetic field gradients according to a predetermined imaging protocol;
d. receiving RF imaging signals generated in said RF receiver coil by said excited nuclear magnetization;
e. reconstructing a magnetic resonance image of said volume of interest from the received magnetic resonance imaging signals and from said position of said RF receiver coil; and,
f. transmitting a signal from each of at least one of said plurality of detectors to a controller located external to said volume, said transmission commencing at a predetermined time relative to the commencement of step (c) and continuing for a predetermined length of time.

114. The method of claim 111, wherein said at least one other imaging technique is selected from the group consisting of (a) fluorescence spectroscopy; (b) SPECT; (c) PET; and
(d) any combination thereof.

115. A method for hyperpolarizing un-polarized gas within an animal, comprising steps of:
a. providing said animal at least partially containing said un-polarized gas;
b. obtaining hyper polarization means for hyperpolarizing said un-polarized gas;
c. hyperpolarizing said un-polarized gas;
wherein said step of hyperpolarizing said un-polarized gas is performed in situ within said animal.

116. The method according to claim 115, additionally comprising at least one step selected from a group consisting of (a) selecting said hyper polarization means is selected from laser, ultrasound, RF, microwave, application of heat or any combination thereof; (b) selecting said gas is selected from helium or Xenon; (c) selecting said animal is selected from a group consisting of mammal, premature babies, humans, reptiles, sea animals, biological specimens, biological organs, mice, rats, rodents, birds, reptiles, amphibians, in vivo biological tissue or organ or ex vivo biological tissue or organ; and any combination thereof.

117. A method for hyperpolarizing un-polarized gas confined within a volume, said volume having a medium therein, said method comprising steps of:
a. providing at least one volume confining an un-polarized gas and at least one medium;
b. obtaining hyper polarization means for hyperpolarizing said un-polarized gas;
c. hyperpolarizing said un-polarized gas;
wherein said step of hyperpolarizing said un-polarized gas is performed in vitro within said confined volume.

118. The method according to claim 117, additionally comprising at least one step selected from a group consisting of (a) selecting said hyper polarization means is selected from laser, ultrasound, RF, microwave, application of heat or any combination thereof; (b) selecting said gas is selected from helium or Xenon; (c) providing a chamber in fluid communication with said volume, said chamber accommodating at least one animal, such that said hyperpolarized gas is supplied from said volume to said chamber; (d) selecting said animal from a group consisting of mammal, humans, premature babies, reptiles, sea animals, biological specimens, biological organs, mice, rats, rodents, birds, reptiles, amphibians, in vivo biological tissue or organ or ex vivo biological tissue or organ.

119. A method for hyperpolarized gas imaging of at least one animal, said method comprising steps of:
a. providing at least one chamber confining a volume in size and shape;
b. accommodating said at least one animal within said at least one chamber;
c. providing at least one volume confining an un-polarized gas and at least one medium;
d. supplying un-polarized gas to said at least one chamber;
e. hyperpolarizing said un-polarized gas; and,
f. imaging at least a region of said animal whilst at least one region of said animal contains said hyperpolarized gas for at least a portion of the time required for said imaging;
wherein said step of hyperpolarizing said un-polarized gas is performed in vitro within said confined volume.

120. The method according to claim 119, additionally comprising at least one step selected from a group consisting of (a) selecting said imaging device from a group consisting of NMR, MRI, CT, X-ray, ultrasound device, fluorescence device, thermographic device or any combination thereof; (b) selecting said hyperpolarization means from laser, RF, ultrasound, microwave, application of heat or any combination thereof; (c) selecting said animal from a group consisting of mammal, humans, premature babies, reptiles, sea animals, biological specimens, biological organs, mice, rats, rodents, birds, reptiles, amphibians, in vivo biological tissue or organ or ex vivo biological tissue or organ; (d) selecting said gas from helium or Xenon; (e) pausing said hyperpolarizing of said un-polarized gas during said step of imaging; (f) selecting said medium from a group consisting of anesthetic gas, water, oxygen or any combination thereof; (g) supplying said chamber with anesthetic gas, water, oxygen or any combination thereof; and any combination thereof.

121. The system according to claim 93, wherein at least one of the following is being held true (a) said medium is selected from a group consisting of anesthetic gas, water, oxygen or any combination thereof; (b) anesthetic gas, water, oxygen or any combination thereof is supplied to said chamber; (c) said imaging device is selected from a group consisting of NMR, MRI, CT, X-ray, ultrasound device, fluorescence device, thermographic device or any combination thereof; (d) said hyper polarization means is selected from laser, ultrasound, microwave, RF, application of heat or any combination thereof; (e) said gas is selected from helium or Xenon; (f) said animal is selected from a group consisting of mammal, humans, premature babies, reptiles, sea animals, biological specimens, biological organs, mice, rats, rodents, birds, reptiles, amphibians, in vivo biological tissue or organ or ex vivo biological tissue or organ.

122. The system of claim 101, wherein the coupled imaging system (CIS) provides an imaging method selected from the group consisting of (a) fluorescence spectroscopy, (b) SPECT, (c) PET, and any combination of the above; and further wherein said plurality of either detectors and/or optics fibers are adapted for detecting signals typical of said at least one additional imaging method.
